(19) United States
(12) Patent Application Publication
(10) Pub. No.: US 2023/0402174 A1
(43) Pub. Date: Dec. 14, 2023

(54) MEDICAL DEVICE USER CONTROL SYSTEM WITH AUTOMATIC SURGEON ANTI-STAGNATION SYSTEM

(71) Applicant: Intuitive Surgical Operations, Inc., Sunnyvale, CA (US)

(72) Inventor: Russell L.E. Blanchard, San Jose, CA (US)

(21) Appl. No.: 18/034,644

(22) PCT Filed: Nov. 2, 2021
(86) PCT No.: PCT/US2021/057688

§ 371 Date: Apr. 28, 2023

Related U.S. Application Data
(60) Provisional application No. 63/108,746, filed on Nov. 2, 2020.

(51) Int. Cl. G16H 40/67; A61B 90/60

(52) U.S. Cl. CPC G16H 40/67; A61B 90/60

(57) ABSTRACT

An example teleoperated surgery user control system includes a first user control and a controller. The first user control is configured to be used by a user to teleoperate one or more aspects of a surgical system. The first user control includes one or more portions configured to articulate and to support one or more body portions of a user. The controller is communicatively connected to the first user control. The controller is configured to execute instructions to automatically articulate the one or more portions periodically or continually during a surgical procedure teleoperated by the user with the surgical system.

### CLAIM OF PRIORITY

This application claims the benefit of priority to U.S. Provisional Patent Application No. 63/108,746, filed on Nov. 2, 2020, which is incorporated by reference herein in its entirety.

### TECHNICAL FIELD

This document relates generally to medical devices, and more particularly, to user control systems and methods for teleoperated surgical systems.

### BACKGROUND

Surgical systems, such as those employed for minimally invasive and orthopedic medical procedures, can include large and complex equipment to precisely control and drive relatively small instruments. Such systems are sometimes referred to as a teleoperated systems or robotic surgical systems. One example of a teleoperated surgical system is the da Vinci® Surgical System commercialized by Intuitive Surgical, Inc.

Teleoperated systems can control and drive multiple instruments through one or more access ports in the body of the patient. Each instrument is typically configured for articulation and to perform various medical tasks (e.g., grasping, cutting, et cetera), and they are controlled by a clinician through a sophisticated computer-assisted user control system.

Among many other functions, the user control system is configured to translate movements of one or more input controls of the user control system by the clinician into movements of the corresponding one or more instruments of the surgical system. During the course of a procedure, however, the clinician may become fatigued or may experience some level of physical discomfort and physical stagnation by being poised in a constant position at the user control system to operate the instruments of the surgical system for a long time. Such fatigue and discomfort become especially serious when a clinician performs several procedures during a single day. One option for reducing fatigue and discomfort is for the clinician to take rest breaks from the procedure and move away from the user control system, for example to stretch, take a walk, etc. But, these rest breaks extend the length of the procedure, and minimizing patient time under anesthesia is an important clinical consideration. Therefore, it would be beneficial to have one or more systems incorporated into the user control system to alleviate physical fatigue, discomfort, and stagnation experienced by clinicians during teleoperated surgical procedures.

### SUMMARY

An example teleoperated surgery user control system includes a first user control and a controller. The first user control is configured to be used by a user to teleoperate one or more aspects of a surgical system. The first user control includes one or more portions configured to articulate and to support one or more body portions of a user. The controller is communicatively connected to the first user control. The controller is configured to execute instructions to automatically articulate the one or more portions periodically or continually during a surgical procedure teleoperated by the user with the surgical system.

An example method of operation of teleoperated surgery user control system includes positioning a first user control for use by a user. The first user control is configured to be used by the user to teleoperate one or more aspects of a surgical system. The first user control includes one or more portions configured to articulate and to support one or more body portions of a user. The method also includes automatically articulating, by a controller communicatively connected to the first user control, at least one of the one or more portions periodically or continually during a surgical procedure teleoperated by the user with the surgical system.

An example teleoperated surgery user control system includes a first user control and a processor communicatively connected to the first user control. The first user control includes one or more manipulable input controls for teleoperation of one or more aspects of a surgical system, and one or more body rests configured to articulate and to support portions of a body of a user. The processor is configured to execute instructions to automatically articulate the one or more body rests periodically or continually in micro-increments.

An example telesurgical system control unit includes a movable component contacted by a clinical user during an operation of the control unit by the clinical user to perform a medical procedure, an actuator coupled to move the movable component, and a control system coupled to control the actuator. The control system comprises coded instructions to automatically activate the actuator to move the movable component during the operation of the control unit by the clinical user.

Each of these non-limiting examples can stand on its own or can be combined in various permutations or combinations with one or more of the other examples.

This Summary is intended to provide an overview of subject matter of the present patent application. It is not intended to provide an exclusive or exhaustive explanation of the invention. The detailed description is included to provide further information about various aspects of the inventive subject matter of the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1A is a plan view depicting an example medical system in a surgical environment.

FIG. 1B depicts an example user control system of the teleoperated surgical system of FIG. 1A.

FIG. 1C depicts another example user control system of a teleoperated surgical system.

FIGS. 2A and 2B are elevation and plan views depicting an example teleoperated surgery user control system in accordance with this disclosure.

FIG. 3 depicts an example surgeon console for teleoperation of a surgical system.

### DETAILED DESCRIPTION

An example teleoperated surgery user control system in accordance with this disclosure includes a user control device and a controller. The user control device is configured to be used by a surgeon to teleoperate one or more aspects of a teleoperated surgical system. The user control device can include one or more body rests against which body portions of the surgeon are configured to rest. The body rests are configured to articulate (e.g., in one or more directions and/or through one or more orientations). The controller is communicatively connected to the user control device and configured to automatically articulate the one or more body rests periodically or continually.

Generally, the algorithmic automated motion control of user control devices according to this disclosure may be configured to modulate the position/location/orientation/etcetera of one or more portions of the user control device over time, for example, either periodically or continually. Example control algorithms can, for example, vary the time at which automatic articulations occur, either at a constant or variable frequency, and, additionally, the algorithm(s) can vary the number, sequence, and/or type of articulation that occurs in each movement of the user control device. The control algorithm can execute a set of articulations in series, e.g. one after another in a predefined sequence, and can execute a set of articulations in parallel, e.g. simultaneous movement of multiple portions (e.g., multiple body rests) of the user control device. Additionally, the controller of the user control system can be configured to control motion of multiple user control devices, such automated articulation of multiple devices can be coordinated, e.g. the articulation routine of a first user control device can be algorithmically related to and dependent upon the articulation of a second user control device, or can be executed in separate and unrelated control algorithms.

Automated articulation of user control devices of a teleoperated surgical system can be executed in micro-increments. In some examples, the magnitude of the incremental value of and direction, orientation, etcetera of automated articulation of a user control device may be configured such that the motion is or is nearly imperceptible to the surgeon employing the teleoperated surgery user control system. One way in which such automated motion can be executed to reduce or minimize the perception of such motion by the surgeon is to execute the motion in relatively small increments. For example, each individual articulation may be limited to a maximum value on the order of millimeters versus centimeters (or 1/16 of an inch versus ¼ of an inch). In other examples, a maximum incremental value of each articulation of a user control device may be smaller or greater, depending upon the intended application, including, for example, depending upon user reaction to such automated articulation during a mock or actual surgical procedure.

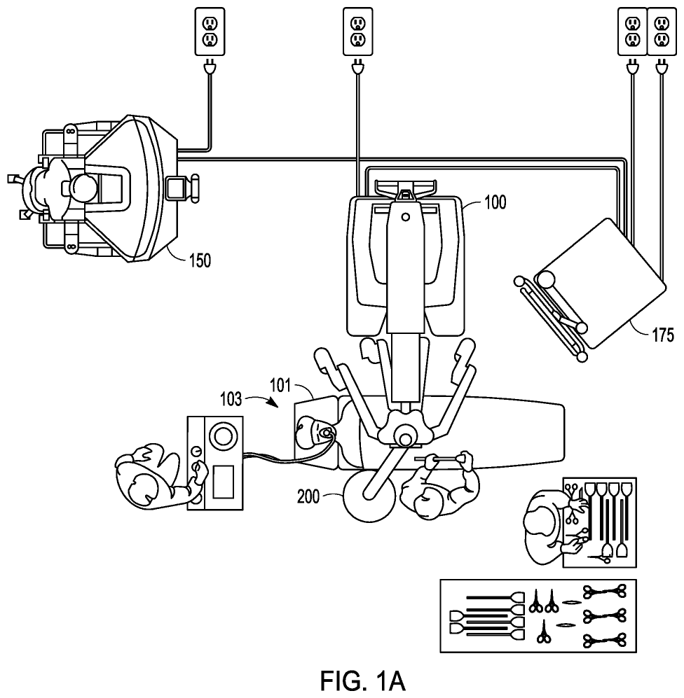

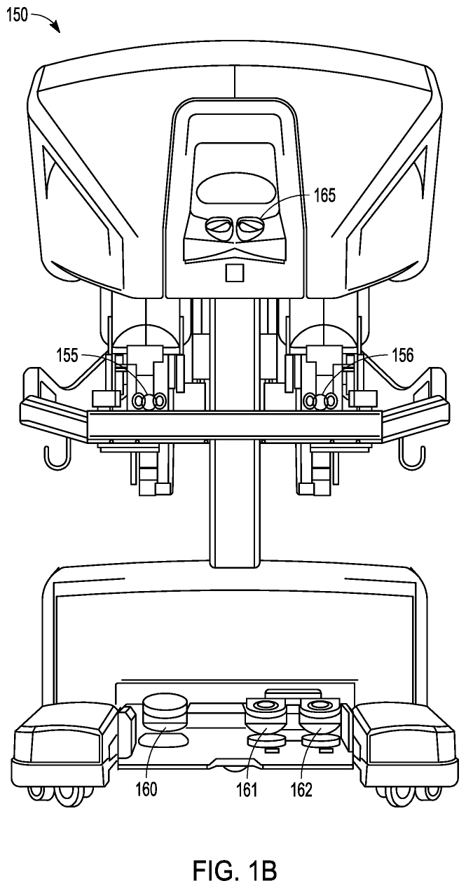

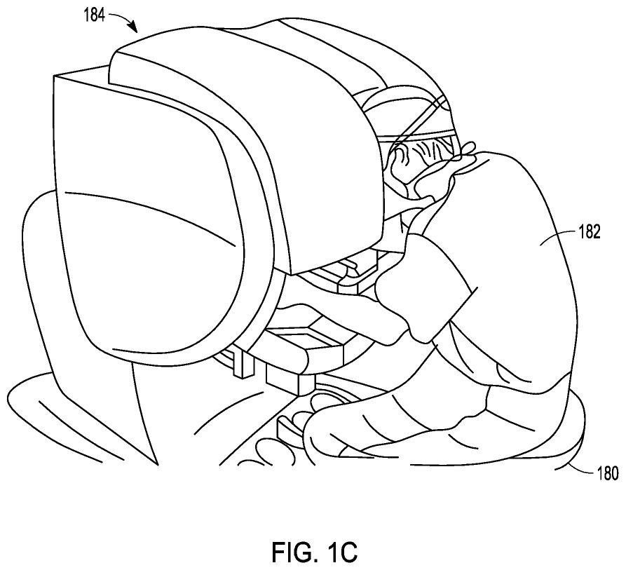

To better understand the environment, systems, and other factors of teleoperated surgery encountered by a surgeon during a procedure, an example teleoperated surgical system and the user control system for the surgical system is depicted and described with reference to FIGS. 1A and 1B.

FIG. 1A is a plan view depicting an example medical procedure environment that includes a multi-arm manipulating system 100 adjacent to a surgical table 101 that supports a patient 103. A second manipulating system 200 may also be situated at the surgical table 101. The manipulating systems 100, 200 may be free-standing on a movable base, or they may be mounted to a table, floor, wall, or ceiling, or they may be supported on another piece of equipment in the clinical environment.

The manipulating system 100 or system 200 may be part of a larger system 10, which may include other sub-systems, including, for example, fluoroscopy or other imaging equipment. One or both of the manipulating systems 100, 200 may be operatively coupled to a user control system 150 or an auxiliary system 175, or both. The user control system 150 (sometimes referred to as user control console and/or surgeon console) may include one or more user input devices (e.g., controls) that may be configured to receive inputs from a user. The user control system 150 may also include or one or more user feedback devices (e.g., viewing system, or tactile or auditory feedback system) that may be configured to provide information to the user regarding the movement or position of an end effector, or an image of a surgical area. The auxiliary system 175 may, for example, include computer processing equipment (e.g., a processor circuit or graphics hardware), or communication equipment (e.g., wired or wireless communication circuits), or endoscopic camera control and image processing equipment.

FIG. 1B depicts example user control system 150. The user control system 150 includes hand controls 155, 156 and foot pedal controls 160, 161, 162. The hand controls 155, 156 and foot pedal controls 160, 161, 162 are used to control equipment at one or more of the manipulating systems 100, 200. For example, an operator may manipulate portions of a distal end of an instrument 130 by using the instrument controls. The controls may include haptic feedback features so that a surgeon may interpret physical information at the instrument 130, such as resistance or vibration, through the controls. The user control system 150 may also include a viewing system 165 that displays video or other images of a surgical site.

In the foregoing examples, one or more of the components in the depicted environment may be considered to form a teleoperated surgical system. For example, and at the least, manipulating systems 100, 200 of FIG. 1A in combination with user control system 150 may form the teleoperated surgical system. Referring again to FIG. 1B, the user control system 150 is generally operated by a surgeon seated in a chair arranged adjacent user control system 150. In some cases, the chair (or other seating device) may be structurally connected to a user control console similar to user control system 150 in FIG. 1B.

FIG. 1C depicts another example user control system, which includes seating device 180, at which surgeon 182 is seated, and surgeon console 184, by which surgeon 182 can execute various surgical procedures using a teleoperated surgical system communicatively connected to console 184. Seating device 180 and console 184 can be ergonomically designed to provide surgeon 182 with proper posture, support, comfort, etcetera. Nevertheless, in the course of a procedure surgeon 182 may become fatigued or may experience some level of discomfort and potentially physical stagnation by being poised in a relatively fixed position at console 184 for extended periods of time. Thus, in examples according to this disclosure, one or more components of the teleoperated surgical system user controls are configured with an automated anti-stagnation system to guard against and decrease the prevalence of surgeon fatigue during teleoperated surgical procedures. Such systems may be beneficial to surgeons or other clinicians to reduce or prevent, for example, neck and/or low back discomfort during teleoperated surgical procedures.

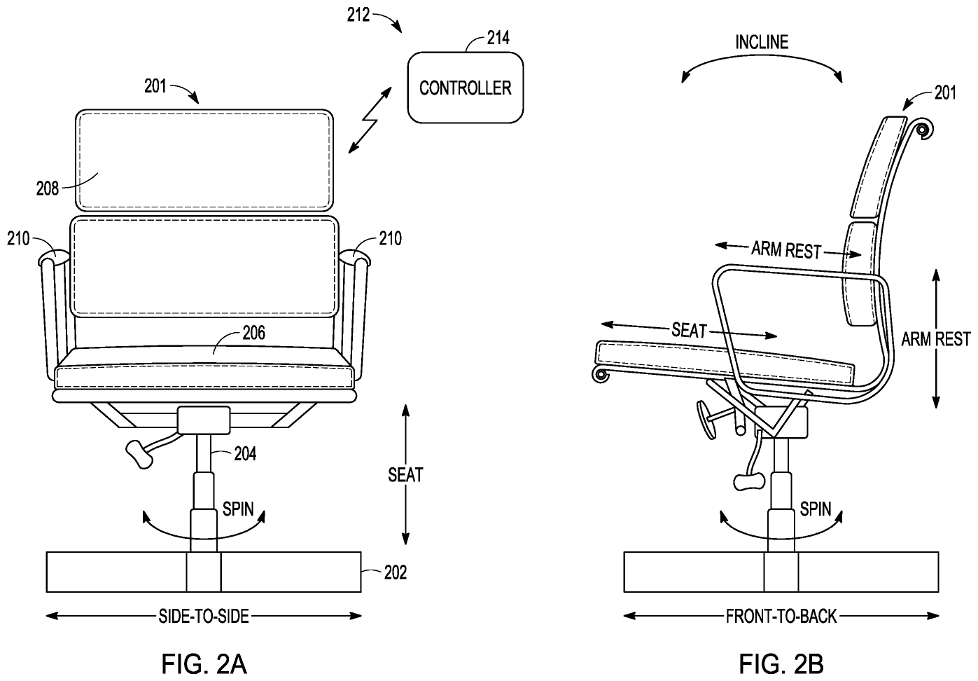

FIGS. 2A and 2B are elevation and plan views depicting an example seating device 201. Seating device 201 may be employed by a surgeon teleoperating a surgical system through a surgical user control console, including example console 184. Seating device 201 includes a system for automatically articulating seating device 201 through one or more degrees of freedom of motion. The seating device 201 can be configured to automatically articulate periodically or continually to modulate the position of the seated surgeon in micro-increments.

In FIGS. 2A and 2B, seating device 201 includes base sled 202, pier 204, seat 206, back rest 208, and arm rests 210. Seating device 201 is supported and articulable in multiple directions by base sled 202. Connected to and protruding up from base sled 202 is one end of support pier 204. Pier 204 can be configured to modulate its own length, which modulation translates into modulation of the height of seat 206 connected to the other end of the pier. Seat 206 is connected to the opposite of end of pier 204 to which base sled 202 is connected, and, as indicated in FIGS. 2A and 2B can be configured to articulate forward and backward. Arm rests 210 are on opposite lateral sides of and connected to seat 206. Each arm rest 210 can be configured to articulate in various ways. For example, arm rest 210 is configured to raise and lower and to slide forward and backward.

Seating device 201 is merely one example of a device of a user control system for a teleoperated surgical system. The manner in which seating device 201 is configured to articulate and the number or character of the directions/orientations through which seating device 201 is configured to move can vary in other examples according to this disclosure. Additionally, the physical configuration of seating device 201 can vary in other examples according to this disclosure. Example seating devices can include stools or other seating configurations that are not a traditional seat with back rest, including example seating device 180 of FIG. 1C. The degrees of freedom of motion of seating device 201 can also vary in other examples according to this disclosure. For example, pier 204 of seating device 201 could be mounted to base sled 202 with more than a simple pivot allowing pier 204 to spin on its long axis. For example, pier 204 could be mounted to base sled 202 with a joint that would yield more complex freedom of motion, including, for example, a spherical or other so-called universal joint.

The mechanical, electro-mechanical, and other devices and systems by which seating device 201 articulates through the various degrees of freedom of motion indicated in FIGS. 2A and 2B can include a variety of types of devices/systems. For example, seating device 201 can include and employ one or more actuators like pneumatic and/or hydraulic cylinders, motors including stepper motors, linear actuators, gears and gear trains, cams, springs, 4 and 6-bar kinematic linkages, as examples, to implement the degrees of freedom of motion of the seating device indicated in FIGS. 2A and 2B.

Regardless of the particular shape, configuration, degrees of freedom of motion, etcetera, seating device 201 and other teleoperated surgery user control devices according to this disclosure are configured to automatically articulate periodically or continually to modulate the position of a seated surgeon in micro-increments, which automated articulation, in turn, is configured to guard against and decrease the prevalence of surgeon stagnation and/or fatigue during teleoperated procedures.

In an example, seating device 201 of FIGS. 2A and 2B is a component of a larger teleoperated surgery user control system 212, which includes seating device 201 and controller 214. In the example of FIGS. 2A AND 2B, controller 214 is depicted as communicatively connected to but physically separate from seating device 201. In other examples, a controller configured for automated control of one or more components of a user control system could be, for example, integrated with and arranged on/in a seating or other user control device employed by a surgeon.

Controller 214 is, as depicted schematically in FIGS. 2A AND 2B, communicatively connected to seating device 201. Controller 214 can include software, hardware, and combinations of hardware and software configured to execute a number of functions related to automatically (e.g., without operator input) articulating seating device 201 in multiple directions and/or through multiple orientations. Controller 214 can be an analog, digital, or combination analog and digital controller including a number of components. As examples, controller 214 can include integrated circuit boards or ICB(s), printed circuit boards PCB(s), processor(s), data storage devices, switches, relays, etcetera. Examples of processors can include any one or more of a microprocessor, a controller, a digital signal processor (DSP), an application specific integrated circuit (ASIC), a field-programmable gate array (FPGA), or equivalent discrete or integrated logic circuitry.

Controller 214 may include storage media to store and/or retrieve data or other information, for example, signals from sensors. For example, seating device 201 may include various devices to provide feedback to controller 214. In an example, electric motors and other devices associated with the multiple degrees of freedom of movement of seating device 201 can be configured to send signals indicative of the current position and/or orientation of seating device 201 to controller 214.

Storage devices of controller 214, in some examples, are described as a computer-readable storage medium. In some examples, storage devices include a temporary memory, meaning that a principal purpose of one or more storage devices is not long-term storage. Storage devices are, in some examples, described as a volatile memory, meaning that storage devices do not maintain stored contents when the computer is turned off. Examples of volatile memories include random access memories (RAM), dynamic random access memories (DRAM), static random access memories (SRAM), and other forms of volatile memories known in the art. The data storage devices can be used to store program instructions for execution by processor(s) of controller 214. The storage devices, for example, are used by software, applications, algorithms, as examples, running on and/or executed by controller 214. The storage devices can include short-term and/or long-term memory, and can be volatile and/or non-volatile. Examples of non-volatile storage elements include magnetic hard discs, optical discs, floppy discs, flash memories, or forms of electrically programmable memories (EPROM) or electrically erasable and programmable (EEPROM) memories.

Controller 214 can be configured to communicate with seating device 201 (and other components of teleoperated surgery user control system 212) via various wired or wireless communications technologies and components using various public and/or proprietary standards and/or protocols. In some examples, controller 214 and other components of user control system 212 will communicate over a local wired communication and/or power network of seating device 201. However, controller 214 can also be configured to communicate wirelessly. Additionally, controller 214 can be configured to use various transport mediums and protocols for communicating with components of system 212, including, for example, Ethernet, Transmission Control Protocol/Internet Protocol (TCP/IP), 802.11 or Bluetooth, or other standard or proprietary communication protocols.

In an example, controller 214 is configured to automatically cause seating device 201 to articulate periodically or continually in micro-increments in accordance with an algorithm or other implementation of a predefined routine or schedule of articulation of the user control device. The algorithm by which controller 214 automatically articulates seating device 201 can be designed to vary the position of the seating device, and thereby the surgeon seated therein, as a function of various variables. For example, controller 214 can periodically execute a predefined series of positional and/or orientation changes of seating device 201 at a constant or varying time frequency. In another example, controller 214 can execute a first set of predefined one or more positional and/or orientation changes of seating device 201 at a first time and can execute a second set of predefined one or more positional and/or orientation changes of seating device 201 at a second time.

Additionally, controller 214 can change the position and/or orientation of seating device 201 based on the state of the teleoperated surgical system. For example, controller 214 can be configured to determine a first operating state of the teleoperated surgical system and to move seating device 201 according to a first predefined schedule during the first operating state of the surgical system. Additionally, controller 214 is configured to determine a first operating state of the teleoperated surgical system and to suspend movement of seating device 201 according to the predefined schedule during the first operating state of the surgical system. Controller 214 can also resume movement of seating device 201 according to the first predefined schedule after the first operating state of the surgical system. In an example, controller 214 is configured to determine a first operating state of the teleoperated surgical system, suspend movement of seating device 201 according to the predefined schedule during the first operating state of the surgical system, and move seating device 201 according to a second predefined schedule (the second predefined schedule is different than the first predefined schedule) during the first operating state of the surgical system.

Additionally, controller 214 can execute an algorithm that varies both the time at which automated motion of seating device 201 occurs and the type and/or parameters of the automated motion caused by controller 214. For example, controller 214 can execute an algorithm that causes a random set of one or more articulations of seating device 201, which occur at random time intervals. The algorithm (or other set of instructions executable by a controller and/or processor(s) thereof) defining control of motion of seating device 201 by controller 214 can be configured to vary articulation of the user control device according to other variables. For example, controller 214 can be configured to periodically or continually (time) cause seating device 201 to articulate in one or more directions and/or through one or more orientations as a function of the particular surgical procedure being conducted by the surgeon. For example, controller 214 may be configured to execute relatively more articulations as a function of time during more physically strenuous portions of the procedure and relatively fewer articulations during less physically demanding portions of the procedure.

As previously noted, controller 214 is configured to automatically cause seating device 201 to articulate periodically or continually in micro-increments. In some examples, the magnitude of the incremental value of and direction, orientation, etcetera of automated articulation of seating device 201 by controller 214 may be configured such that the motion is or is nearly imperceptible to the surgeon employing the teleoperated surgery user control system. As used in this disclosure, the relative term “nearly imperceptible” can mean that the incremental value by which a portion of a teleoperated surgery user control articulates can be selected to not substantially interrupt any mental or physical processes of the surgeon during the procedure.

One way in which controller 214 can execute automated motion of seating device 201 that minimizes the perception of such motion by the surgeon is to execute the motion in relatively small increments. For example, each individual articulation may be limited to a maximum value on the order of millimeters versus centimeters (or 1/16 of an inch versus ¼ of an inch). In other examples, a maximum incremental value of each articulation of seating device 201 or another user control device may be smaller or greater, depending upon the intended application, including, for example, depending upon user reaction to such automated articulation during a mock or actual surgical procedure.

Additionally, although some of the foregoing examples include periodic or episodic execution of automated articulation of seating device 201, in some examples, controller 214 may be configured to continually and constantly vary the position and/or orientation of the seating device in difficult to perceive micro-increments to yield the effect that the seating device or other user control device is breathing or continually slightly refreshing the position/orientation/etcetera of the surgeon employing the device.

Controller 214 can execute each individual articulation of seating device 201 (or other user control device) in series or in parallel with one another. For example, controller 214 can be configured to cause seat 206 of seating device 201 to change vertical position, and, thereafter, cause one or both of arm rests 210 to change forward/backward position. In another example, controller 214 can be configured to simultaneously 1) cause seat 206 of seating device 201 to change vertical position, and 2) cause one or both of arm rests 210 to change forward/backward position.

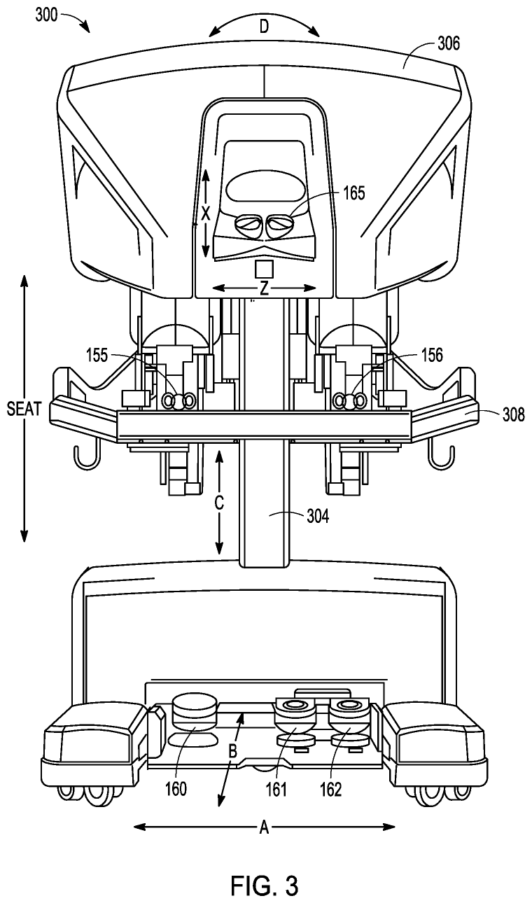

FIG. 3 depicts example surgeon console 300 for teleoperation of a surgical system. Console 300 can be similar to console 150 of FIG. 1B. For example, console 300 can include hand controls 155, 156 and foot pedal controls 160, 161, 162. The hand controls 155, 156 and foot pedal controls 160, 161, 162 are used to control one or more operational or other aspects of a teleoperated surgical system. For example, an operator may manipulate portions of a distal end of an instrument of the surgical system by using the instrument controls. The controls may include haptic feedback features so that a surgeon may interpret physical information at the surgical instrument(s), such as resistance or vibration, through the controls. Additionally, console includes viewing system 165 that displays video or other images of a surgical site. Although console 300 includes only “hand” and “foot” controls 155, 156 and 160, 161, 162, another example user control according to this disclosure can include other surgeon-manipulable input controls, including, for example, head, finger, thumb, and tongue-manipulable input controls.

FIG. 3 depicts a plurality of articulations/degrees of freedom of motion of console 300 at direction/orientation arrows A-G. These are only example articulations of console 300 and, in other examples, a teleoperated surgery user control device according to this disclosure may include more, fewer, and/or different articulation capabilities. Additionally, console 300 can be communicatively connected to a controller like controller 214, which controller can be configured to execute one or more algorithms for automated periodic or continuous articulation of surgeon console 300. Surgeon console 300 is thus an example of a teleoperated surgery user control device other than a seating device, which may be configured to automatically modulate the position of a surgeon in micro-increments, which automated articulation, in turn, is configured to guard against and decrease the prevalence of surgeon stagnation and/or fatigue during teleoperated procedures. One unifying feature/principle of example seating device 201 and example surgeon console 300 is the ability to automatically modulate surgeon seating/standing position on a device on/against which a portion of the surgeon's body is configured to engage/rest.

For example, viewing system 165 can include a forehead rest/pad, against which the surgeon is configured to rest their head during a teleoperated surgical system. This point/location of engagement with the surgeon's body is quite distinct from those of seating device 201, and, yet, the principle of automated periodic or continuous articulation of portions of viewing system 165 are the same or substantially similar. In the example of FIG. 3, foot pedals 160-162 can be configured to slide laterally in direction A and forward/backward (or, from the perspective of FIG. 3, in and out of the page) in direction B. The articulation of foot pedals 160-162 can be independent of and relative to sliding or other movement of base 302 of console 300. Additionally, pier 304 of console 300 may be configured to raise and lower the upper portion of console 300 in direction C, and hood 306 of console 300 or pier 304 can be configured to rotate hood 306/upper portion of console 300 about a long axis of pier 304 in direction/rotational orientation D.

When operating hand controls 155 and 156, the surgeon may rest their arms on bar 308, which is coupled to the upper portion of console 300 near a bottom portion of hood 306. In examples, therefore, bar 308 may be configured to automatically periodically or continually articulate up and down, for example, in direction E, as indicated in FIG. 3.

In some examples, seating device 201 and console 300 are included in one system and are controlled by the same or separate communicatively connected controllers to automatically modulate the positions/locations/orientations/etcetera of one or more portions of each of seating device 201 and console 300 via a coordinate movement algorithm (or other executable list of instructions) or in separate, automated movement algorithms for each device. Additionally, a teleoperated surgery user control system in accordance with this disclosure may include additional user control devices other than a seating device and surgeon console.

Persons of skill in the art will understand that any of the features described above may be combined with any of the other example features, as long as the features are not mutually exclusive. All possible combinations of features are contemplated, depending on clinical or other design requirements. In addition, if manipulating system units are combined into a single system (e.g., telesurgery system), each individual unit may have the same configuration of features, or, one patient-side unit may have one configuration of features and another patient-side unit may have a second, different configuration of features.

The examples (e.g., methods, systems, or devices) described herein may be applicable to surgical procedures, non-surgical medical procedures, diagnostic procedures, cosmetic procedures, and non-medical procedures or applications. The examples may also be applicable for training, or for obtaining information, such as imaging procedures. The examples may be applicable to handling of tissue that has been removed from human or animal anatomies and will not be returned to a human or animal, or for use with human or animal cadavers. The examples may be used for industrial applications, general robotic uses, manipulation of non-tissue work pieces, as part of an artificial intelligence system, or in a transportation system.

The above detailed description includes references to the accompanying drawings, which form a part of the detailed description. The drawings show, by way of illustration, specific embodiments in which the invention may be practiced. These embodiments are also referred to herein as “examples.” Such examples may include elements in addition to those shown or described. But, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Geometric terms, such as “parallel”, “perpendicular”, “round”, or “square”, are not intended to require absolute mathematical precision, unless the context indicates otherwise. Instead, such geometric terms allow for variations due to manufacturing or equivalent functions. For example, if an element is described as “round” or “generally round”, a component that is not precisely circular (e.g., one that is slightly oblong or is a many-sided polygon) is still encompassed by this description. Coordinate systems or reference frames are provided for aiding explanation, and implantations may use other reference frames or coordinate systems other than those described herein.

The above description is intended to be illustrative, and not restrictive. For example, the above-described examples (or one or more aspects thereof) may be used in combination with each other. Other embodiments may be used, such as by one of ordinary skill in the art upon reviewing the above description. The Abstract is provided to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments may be combined with each other in various combinations or permutations. The scope of the invention should be determined with reference to the appended claims, along with the full scope of equivalents to which such claims are entitled.

1-60. (canceled)

61. A teleoperated surgery user control system comprises:
a first user control comprising:
one or more manipulable input controls for teleoperation of one or more aspects of a surgical system; and
one or more body rests configured to articulate and to support portions of a body of a clinical user of the surgical system; and
a processor communicatively connected to the first user control, the processor configured to execute instructions to automatically articulate the one or more body rests periodically or continually in micro-increments, wherein:
the automatic articulation includes changing one or both of a location and an orientation of the one or more body rests; and
the magnitude of each micro-increment is selected so that articulation is functionally imperceptible to the clinical user.

62. The system of claim 61, wherein the first user control comprises one or more manipulable input controls for teleoperation of the one or more aspects of the surgical system.

63. The system of claim 62, wherein the one or more manipulable input controls comprises at least one of a hand, foot, head, finger, thumb, and tongue-manipulable control.

64. The system of claim 61, wherein each of the one or more body rests is configured to:
translate in one or more directions; and/or
rotate in one or more directions.

65. The system of claim 61, wherein the first user control comprises one or more actuators configured to articulate the one or more body rests.

66. The system of claim 65, wherein the one or more actuators comprises at least one of a motor, a linear and/or rotary actuator, one or more gears, one or more cams, one or more springs, and a kinematic linkage.

67. The system of claim 61, wherein the controller is configured to:
articulate a first body rest of the one or more body rests at a first time; and
articulate a second body rest of the one or more body rests at a second time.

68. The system of claim 67, wherein the first time is different than the second time.

69. The system of claim 61, wherein the controller is configured to:
simultaneously articulate a first plurality of the one or more body rests at a first time; and
simultaneously articulate a second plurality of the one or more body rests at a second time.

70. The system of claim 61, wherein the controller is configured to:
sequentially articulate a first plurality of the one or more body rests commencing at a first time; and
sequentially articulate a second plurality of the one or more body rests commencing at a second time.

71. The system of claim 61, wherein the controller is configured to articulate at least one of the one or more body rests periodically over time at a constant frequency.

72. The system of claim 61, wherein the controller is configured to articulate at least one of the one or more body rests periodically over time at a variable frequency.

73. The system of claim 61, wherein the controller is configured to articulate at least one of the one or more body rests periodically over time at a random frequency.

74. The system of claim 61, wherein the controller is configured to simultaneously articulate a plurality of the one or more body rests.

75. The system of claim 61, wherein the controller is configured to sequentially articulate a plurality of the one or more body rests.

76. The system of claim 61, wherein the controller is configured to articulate at least one of the one or more body rests at a speed functionally imperceptible to the clinical user.

77. The system of claim 61, wherein the controller is configured to articulate at least one of the one or more body rests at a frequency functionally imperceptible to the clinical user.

78. The system of claim 61, wherein the controller is configured to articulate at least one of the one or more body rests on a predefined schedule.

79. The system of claim 78, wherein:
the predefined schedule comprises at least one of:
articulation of the at least one of the one or more body rests continually;
articulation of the at least one of the one or more body rests at a constant frequency;
articulation of the at least one of the one or more body rests at a variable frequency; and
articulation of the at least one of the one or more body rests at a random frequency.

80. The system of claim 61, wherein the one or more body rests comprise at least one of:
a seat on which the clinical user sits during use of the first user control;
an armrest on which the clinical user rests an arm during use of the first user control;
a foot rest on which the clinical user rests a foot during use of the first user control;
a viewing system having a pad against which the clinical user is configured to rest their head during use of the first user control;
a hand-operated control input with which the clinical user controls movement of an instrument of the surgical system via the first user control.

81. A telesurgical system control unit comprising:
a movable component contacted by a clinical user during an operation of the control unit by the clinical user to perform a medical procedure;
an actuator coupled to move the movable component; and
a control system coupled to control the actuator;
wherein the control system comprises coded instructions to automatically activate the actuator to move the movable component during the operation of the control unit by the clinical user, wherein:
the actuator moves the movable component by changing one or both of a location and an orientation thereof, and
the actuator moves the movable component at a frequency functionally imperceptible to the clinical user.
